(19) United States
(12) Patent Application Publication
(10) Pub. No.: US 2004/0122341 A1
(43) Pub. Date: Jun. 24, 2004

(54) Luminal directional force measurement and electrical stimulation probe

(76) Inventors: Edward G. Walsh, Irondale, AL (US); Vicki Young Johnson, Hoover, AL (US)

Correspondence Address: Benjamin Aaron Adler, ADLER & ASSOCIATES, 8011 Candle Lane, Houston, TX, 77071, US

(21) Appl. No.: 10/684,118

(22) Filed: Oct. 10, 2003

Related U.S. Application Data
(60) Provisional application No. 60/417,552, filed on Oct. 10, 2002.

(51) Int. Cl. A61B 5/103

(52) U.S. Cl. 600/591

(57) ABSTRACT

Provided herein is a luminal force measurement device to assess pelvic floor muscle function in an individual for diagnosis or treatment thereof comprising a cylindrical probe having a section insertable into a body lumen, where the insertable section is disposable or is non-disposable, and a non-disposable section connected thereto; a plurality of force transducers; a plurality of stimulation electrode pairs; a plurality of differential instrumentation amplifiers; and a means of securing the probe into a holder. Also provided are methods for use of the device.

### CROSS-REFERENCE TO RELATED APPLICATIONS

This non-provisional application claims benefit of provisional U.S. Serial No. 60/417,552, filed Oct. 10, 2002, now abandoned.

### BACKGROUND

1. Field of the Invention

The present invention relates generally to the fields of medical devices, medical diagnostics and treatment. More specifically, the present invention relates to a luminal pressure transduction device for time-resolved and direction-resolved measurement of regional contractile effort. Additionally, the present invention provides for regional electrical stimulation, and for the ability to perform regional electrical stimulation with simultaneous regional pressure measurement.

2. Description of the Related Art

Exercise, for reconditioning the pelvic floor muscles, has been used as a therapeutic intervention in the treatment of urinary and fecal incontinence. Specificity of training is paramount in achieving optimal function. In particular, muscles of the levator ani are involved in the maintenance of urinary continence. Kegel (1) introduced pelvic floor musculature exercises with a reported 69-93% success rate in treating females with stress urinary incontinence (SUI) (2-5). Investigators have hypothesized the mechanism for improvement as being exercise-induced hypertrophy because studies to describe the pelvic floor musculature in regard to muscle fiber type and mechanism of action have been limited to in vivo biopsy at the time of surgery or cadaver dissection (6).

Although there have been many advancements in the treatment of urinary incontinence using pelvic floor muscle exercises within a behavioral framework, investigators have been unable to describe the precise mechanisms of improvement. There are many potential and competing theories for the mechanisms of action responsible for recovery of continence. Some have hypothesized that increasing muscle strength allows the patient better sphincter control. Others have suggested that with exercise, the muscle size increases providing additional occlusive bulk around the urethral sphincter.

In cases where incontinence has followed vaginal childbirth, the role of nerve injury or scarring, i.e., collagen deposition, has been targeted as playing a role in loss of urine control. Likewise, the type of collagen found in the pelvic floor has recently come into scrutiny as a contributor to pathologic pelvic relaxation. There is little agreement on the correct technique for performing pelvic floor musculature exercise (7) and few studies have been undertaken to determine contraction intensity level of exercise to ensure success in pelvic floor muscle exercise therapy (8).

Urinary incontinence in women is a consequence in part of partial or complete failure in the function of the supportive muscles of the pelvic floor. Advancements have been made in the assessment and therapy to repair or recondition the pelvic floor muscles for restoration of urinary continence. Urinary incontinence is the result of factors such as failure to store, failure to evaluate, sensory deficits, or a combination of the three. Loss of muscle function can result from injuries as a consequence of childbirth, neurological disorders, i.e., multiple sclerosis or physiologic changes attributed to aging. While no one therapy is appropriate for all types of incontinence, it is paramount that assessment of the pelvic floor function be carried out in an accurate manner to permit selection of the most appropriate therapy. This assessment, ideally, should include contractile function with spatial resolution to permit isolation of muscle groups exhibiting poor function or dysfunction.

Measurement of contractile force is currently performed using either devices in which pressure is measured using a balloon-type pneumatic pressure transducer or by collecting electromyogram (EMG) signals to estimate muscular activity. In the case of the pneumatic measurement the information obtained represents an averaged measurement with no directional information provided. The EMG provides information on electrical activity only and contains an element of ambiguity owing to “crosstalk” between muscle groups. It is important to generate spatially-resolved information on the mechanical function of the pelvic floor muscles in regard to their contribution in attaining and maintaining urinary continence. In the case of an asymmetric muscular response generalized exercise therapy could result in further strengthening of normally functioning muscles, while weak muscle(s) sustain further damage as the result of excessive strain produced by the stronger muscles.

A corollary to current pelvic floor muscle exercise therapy is electrical stimulation. In transvaginal, transrectal and perianal skin patch electrode electrical stimulation a stimulus is delivered and intended to evoke excitation of the pelvic floor muscles to induce a generalized passive or involuntary contraction. The stimulus effect has been measured indirectly in terms of the amount of stimulus required to excite the muscle, i.e., 100 Hz, and subjectively as described by subjects.

The prior art is deficient in a luminal directional force measurement device to assess regional pelvic floor muscle function. Additionally, the prior art is deficient in a directional luminal force measurement device to apply electrical stimuli to selected regions of the pelvic floor musculature. Furthermore, the prior art is deficient in a luminal directional force measurement device to assess developed contraction force in response to electrical stimulus to specific regions of the pelvic floor musculature. The present invention fulfills these longstanding needs and desires in the art.

### SUMMARY OF THE INVENTION

In the present invention, stimuli are targeted to excite a specific region(s) of the pelvic floor musculature, such as the posterior center, left anterior, etc., and the muscular response will be concomitantly quantitatively measured in terms of developed force. This unique capability permits isolation and stimulation of dysfunctional regions of the pelvic floor musculature.

The present invention is directed to a luminal force measurement device to assess pelvic floor muscle function for diagnosis or for treatment of an individual. The device comprises a cylindrical probe having a disposable or a nondisposable section insertable into a body lumen, which contains a plurality of force transducers and a plurality of stimulation electrode pairs, and a non-disposable section connected thereto which contains a plurality of differential instrumentation amplifiers. The device has a means of holding the probe in position within the body lumen. The device further comprises an interface unit containing additional amplification, analog-to-digital conversion and a computer for data collection, acquisition control, data analysis, and display, as well as a means for archiving data.

The present invention is further directed to a method of using the luminal force measurement device described herein to assess pelvic floor muscle function in the individual.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1A depicts individual force transducer elements.

FIG. 1B depicts a probe with disposable sheath detailing the transducer and electrode placement.

FIG. 2A depicts the probe mounting platform and holder.

FIG. 2B depicts the mechanism for tracking angular probe displacement.

FIG. 3A depicts a transducer differential amplifier circuit diagram.

FIG. 3B depicts an interface block diagram.

FIG. 4 depicts time-pressure curves and derivatives thereof.

### DETAILED DESCRIPTION OF THE INVENTION

In one embodiment of the present invention, there is provided a luminal force measurement device to assess pelvic floor muscle function for diagnosis or for treatment of an individual comprising a cylindrical probe having a section insertable into a body lumen, where the insertable section is disposable or is non-disposable, and a non-disposable section connected thereto; a plurality of force transducers; a plurality of stimulation electrode pairs; a plurality of differential instrumentation amplifiers; and a means of holding the probe in position within the body lumen.

Further to this embodiment, the luminal force measurement device can further comprise an interface unit which has a second stage of force transducer signal amplification; gain and DC offset adjustment; electrical stimulation signal generation; analog-to-digital conversion; and a means for connecting the probe to a control computer. Examples of means of connecting the interface to the computer are a standardized serial port, a standardized parallel port, a standardized wired communication path or a standardized wireless communication path. Additionally the interface unit may be battery powered and the control computer may be a battery powered portable computer.

In an aspect of this embodiment the means to hold the probe in position may comprise an examination platform and a probe holder detachable therefrom. In this aspect the non-disposable section of the probe may connect to the probe holder. Further in this aspect the examination platform has two sections that are hinged with handles at either end for portability. One section comprises the detachable probe holder. The examination platform may be padded on the upper surfaces. The examination platform may be positioned on a bed, an examining table or mounted on the wall for use in examining subjects in a standing position. Additionally, the probe holder may be detached prior to transporting the examination platform. The probe holder also allows for adjustment of the position, i.e., tilt and penetration of the probe within the individual prior to securing the probe within the holder.

In another aspect of this embodiment the means to hold the probe in position is a multiposition probe stabilizer comprising a probe base connection housing which has a connector for the probe and axial displacement sensors connected to the probe to detect displacement of the probe from an axis along the length of the probe when the probe is inserted within a body lumen. In this aspect the non-disposable section of the probe connects to the connector in the probe base connection housing. Further to this aspect the probe stabilizer may comprise a calibrated spring system resistant to the axial displacement of the probe within the body lumen.

Additionally, in this aspect the probe stabilizer may comprise a connection housing holder which attaches to the probe base connection housing. The holder comprises a cross-member having a spring therein to which the connection housing may attach and a support structure at each end of the housing support where each of the support structures has a surface against which an inner thigh of an individual is positioned. Furthermore, the holder may comprise a removable pad disposed between the supporting structure and the inner thigh of the individual.

In related aspects, the force transducers and pairs of stimulation electrodes are contained on a disposable sheath such that each of the electrode pairs is in proximate relationship to one of the force tranducers. The disposable sheath removably covers the non-disposable insertable section of the cylindrical probe. Alternatively, the force transducers and the stimulation electrode pairs are permanently affixed to the insertable section of the probe such that each of the electrode pairs are in proximate relationship to one of the force tranducers. In this aspect the entire insertable section is disposable. Further to either of these aspects, the force transducers and the pairs of stimulation electrodes are disposed annularly in a single row or in multiple rows around the insertable section of the probe.

In all aspects of this embodiment, force transducers may be strain gauges, optical sensors, piezoelectric sensors, pneumatic mechanisms, or resistive/pressure sensors. Optionally, the stimulation electrodes may be used electromyography electrodes simultaneously with pressure measurements. The force transducers have a directional component for measurable radial force. The differential instrumentation amplifiers may be located on the non-disposable section of the probe.

Also in all aspects the probe may have depth index markings on the disposable insertable section or on a sheath covering the non-disposable insertable section indicating depth of insertion into the body lumen. The insertable section of the probe may comprise a rigid or a compliant material. The probe may have a total length of at least 20 cm. Further, the tip of the insertable section of the probe is rounded.

In these aspects the probe may be inserted intravaginally or intrarectally. The probe may have a diameter of about 1.5 cm to about 2.5 cm where the force transducers and the stimulation electrodes are positioned about 5 cm from the tip of the insertable section of the probe. Alternatively, the probe may have a diameter of about 0.75 cm to about 1.5 cm where the force transducers and the stimulation electrodes are positioned about 2 cm from the tip of the insertable section of the probe.

In another embodiment of the present invention, there is provided a method of using the luminal force measurement device to assess pelvic floor muscle function comprising the steps of positioning a probe of the luminal force measurement device described supra within an individual intravaginally or intrarectally; causing pelvic muscles to generate force; transducing the developed muscular force to an electrical signal; converting said electrical signal from an analog signal to a digital signal; transmitting the digital signal to a control computer; and converting the signal to data thereby assessing the pelvic floor muscle function in the individual. In this embodiment all aspects of the luminal force measurement device are as described supra.

In an aspect of this embodiment the force may be applied voluntarily by the individual via contraction of the pelvic floor muscles or the force may be produced by an electrical stimulus applied to specific regions. In this aspect termination of the contractile force by voluntary relaxation or by cessation of electrical stimulus to the specific regions is monitored to assess quantitatively the rate of relaxation and uniformity in the relaxation rate.

The present invention provides an insertable device used as a vaginal or rectal probe for the purpose of providing directional and time resolved information on contraction effort for the purpose of regional assessment of pelvic floor muscle function as would be suitable, for example, for use in diagnosis and treatment of urinary incontinence in women. In addition, a mechanism is provided for delivering regional electrical stimulus with provision for obtaining simultaneous regional pressure measurements during stimulation to assess actual muscular response. These capabilities are lacking in the current devices used for pelvic floor muscle assessment and rehabilitation.

The luminal directional force probe for intravaginal or intrarectal use can comprise a cylindrical device which contains two or more force transducers on the probe surface that provide for measurement of force perpendicular to the axis of the device. Contraction force can be measured at multiple locations. The probe can be sterilizable and non-disposable with the force transducers and stimulation electrodes contained within a disposable covering or sheath. The disposable sheath contains the stimulation electrodes in pairs with the electrode pairs co-located with the force transducers to provide for simultaneous delivery of electrical stimulation and regional contractile force measurement.

Alternatively, the device can also comprise a disposable section containing multiple force transducers and pairs of stimulation electrodes permanently affixed to the probe surface. This disposable section connects to the non-disposable portion of the probe which contains the instrumentation amplifiers. In this embodiment, no sterilization is necessary. The force transducer and electrode distribution is as with the disposable sheath.

The probe may comprise a rigid or compliant material. The compliant material compresses in response to pressure exerted upon the probe. The probe, however, remains fixed in position to provide directional pressure measurements. The material returns to its original shape when pressure is released. The probe may contain index markings to provide for known and/or repeatable depth of insertion and to verify proper location of the pressure or force transducers and stimulus electrodes. The markings may be on a disposable sheath covering a non-disposable insertable section of the probe or may be on the disposable insertable section of the probe.

The diameter of the cylinder is established to be appropriate for the anatomy to be examined. In the case of examination of women with urinary or fecal incontinence the cylinder will range in diameter from approximately 0.75 cm to 2.5 cm depending on intended location of use, i.e., intravaginally or intrarectally, and individual anatomic variation. The length of the probe is sufficient to locate the force transducers and stimulation electrodes between 1.5 and 5 cm from the tip of the probe while providing for a section of the probe body where it is attached to a restraining mechanism. The total length of the probe is sufficient to provide for proper insertion and for locking of the probe into a holder. The total length of the probe may be, but is not limited to, at least 20 cm.

The force transducers may be arranged in a single annular region or can contain multiple annular regions permitting not only radial resolution of force direction, but depth resolution as well along the long axis. These transducers may be flat resistive sensors, piezoelectric sensors, optical sensors, strain gauges, or pneumatic transduction mechanisms. It is also possible to transmit mechanical force to strain gauges or pressure transducers located away from the sensing region of the device.

Stimulation electrodes are contained on a disposable sheath or are permanently affixed to a disposable portion of the probe and form an annular pattern corresponding to the force transducer locations. There is one pair of stimulation electrodes for each location at which force is measured. The sheath provides for electrical connections via connection points to the non-disposable portion of the probe in order to carry the electrical stimulation signals to the electrodes.

The non-disposable portion of the probe contains differential instrumentation amplifiers to provide a first stage of amplification and to maximize common-mode rejection of electrical noise. The probe connects to an interface unit containing a second stage of force signal amplification, mechanism for DC offset and gain adjustment, analog-to-digital conversion mechanism, electrical stimulation signal source, DC power supply, and appropriate probe and computer connectors. Control of signal acquisition and analysis of force-time data is provided by a computer (desktop or portable) that connects to the interface through a serial or parallel port or through any standardized wired or wireless communication path.

An examination platform is provided which locks the probe position once it is correctly inserted and permits the probe to resolve directional force development. Since the probe is capable of providing directional force information, this restrains the motion of the probe during examination. For example, if a patient develops contractile force on the right side, a freely moving probe will be pressed against the left wall and equal force measurements will be noted from the left and right transducers. By locking the probe position once it is correctly placed, directional force components can be measured, even if asymmetries in force development exist.

Alternatively, the probe is not locked onto a platform, but is attached to a mechanism, which may be a hand-held mechanism, which measures the direction and angular deviation from the reference axis. Such measurement of probe displacement may be time-resolved. The reference axis is defined as the long axis of the probe following insertion while the patient is at rest.

This mechanism provides a calibrated spring resistant to displacement or deviation from the reference axis. This spring constant and angular displacement measurement and the individual pressure measurements from the surface of the probe permit the assessment of regional directional pressure or force which is producing the displacement. With the pressure measurements provided by the force transducers on the probe, directional pressure components may be determined.

This mechanism can be stabilized in a holder by the patient's inner thighs. Additionally, this mechanism permits recording of the path of the probe tip as a subject exercises and relaxes. This mechanism may also contain the first differential amplifier stages for the probe pressure transducers. Signal amplification and sampling are performed as with other embodiments described herein.

In use, either embodiment of the system can be used for diagnostic purposes in which the subject is instructed to execute voluntary contraction efforts of various durations and intensities. Time pressure curves will indicate the performance of the pelvic floor muscle groups responsible for generating force at each transducer location. Such studies can be repeated over time to assess the response of the pelvic floor muscles to therapy such as exercise, electrical stimulation, pharmacologic, or surgical. The regional electrical stimulation feature with simultaneous pressure measurements can be used to assess muscle function exclusive of the subject's ability to voluntarily comply with exercise instructions. Electrical stimulation, as a therapy, can have its efficacy assessed over time as well through use of regional pressure measurements both during stimulation, and during voluntary effort.

Owing to the directional nature of the radial pressure measurements, the present device is capable of distinguishing between a proper contraction and a Valsalva maneuver or thigh/buttock muscle contraction. These latter exercises are inappropriate and do not affect the muscles that provide for continence. If these inappropriate exercises are performed over time there will be no improvement in continence, and possibly the incontinence condition will be worsened.

However, some patients confuse these actions with the proper Kegel-type elevator contraction exercise. The present invention detects these inappropriate exercises by the partial unloading of some of the pressure transducers, observed as a decrease in pressure from resting values. This feature aids in patient identification of the appropriate muscles and in training for exercise therapy.

Additionally, in all embodiments of the instant invention, electromyography (EMG) capability can be included in which the stimulation electrodes are used for electromyography sensing when an electromyography recording device having amplifiers and filters and having output to the A/D system is included in the interface unit.

As described herein, the invention provides a number of therapeutic and diagnostic advantages and uses. Embodiments of the present invention are better illustrated with reference to the Figures, however, such reference is not meant to limit the present invention in any fashion. The embodiments and variations described in detail herein are to be interpreted by the appended claims and equivalents thereof.

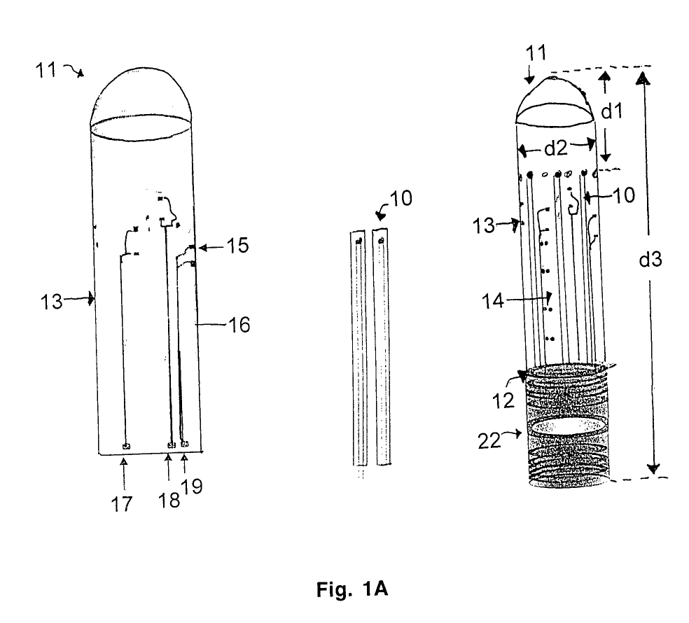

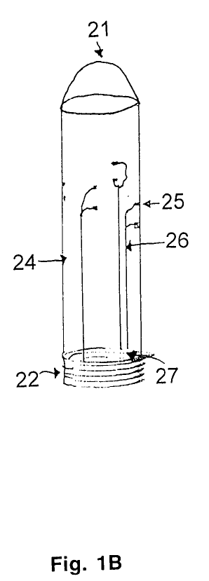

FIG. 1A depicts force transducers 10 placed within disposable covers. The probe 11 comprises an insertable body 14 with the disposable sheath 13 disposed thereon. A locking collar 12 is provided to rigidly attach the disposable sheath 13 to the main insertable body 14 of the probe 11. After use the insertable body 14 can be detached, the sheath 13 removed for appropriate disposal and the insertable body 14 sterilized for further use. The main body 14 contains differential amplifiers (not shown) for the force transducers 10 and provides for locking of the device in a restraining holder. There is a threaded portion on the distal end of the insertable portion, the non-disposable portion and a matching “cap” portion into which the insertable part screws; the insertable part is one piece, the part that houses the amplifiers is non-disposable and separate.

The force transducers 10 are located in an annular pattern on the outer surface of a compliant plastic disposable sheath 13 at an approximate distance d1 from the tip of the probe 11. The distance d1 is approximately 5 cm if the probe 11 is intended for vaginal use and approximately 2 cm if the probe 11 is intended for rectal use. The probe 11 is cylindrical in shape and has a diameter d2. For intravaginal use d2 is about 1.5 cm to about 2.5 cm. For intrarectal use d2 is about 1 cm to 1.5 cm. Furthermore, the probe 11 has a total length d3 of about 25 cm. This provides for adequate penetration depth while allowing for the probe to be clamped or fixed in position. The tip of the main body 14 may be outwardly rounded for ease of insertion of the probe 11. Force transducers 10 provide for perpendicular force measurement, as referenced to the probe surface, at six locations for example.

Stimulation electrodes 15 are embedded in the outer surface of the sheath 13 such that the electrodes 15 can make electrical contact with the mucosa. Conductors 16 each have a first end that are connected to the stimulation electrodes 15 and are insulated from contact with tissue. The sheath 13 also provides for electrical connection points 17, 18, 19 to the non-disposable portion of the probe (not shown) to which a second end of each of the conductors 16 is connected. The flexible nature of the sheath provides for force transmission to the force transducers during contraction.

FIG. 1B depicts the disposition of stimulation electrode pairs in a probe in which the entire insertable portion of the probe is disposable. Pairs of stimulation electrodes 25 are provided and are located near each force transducer (not shown) all of which are permanently affixed to the insertable body 24 of the probe 21 to provide for regional electrical stimulation at locations corresponding to locations of force measurement. The conductors 26 connected to the electrodes 25 at a first end are insulated from contact with tissue to insure that stimulation is delivered only to the locations corresponding to force transducer positions. The conductors 26 are attached by each of a second end to the non-disposable part of the probe (not shown) through drilled holes 27 in the probe casing then sealed with epoxy. The disposable probe 21 has a locking device 22 to detachably affix the disposable probe body 24 to the non-disposable section (not shown) of the probe 21. After use the insertable section of the probe 24 is detachable for appropriate disposal.

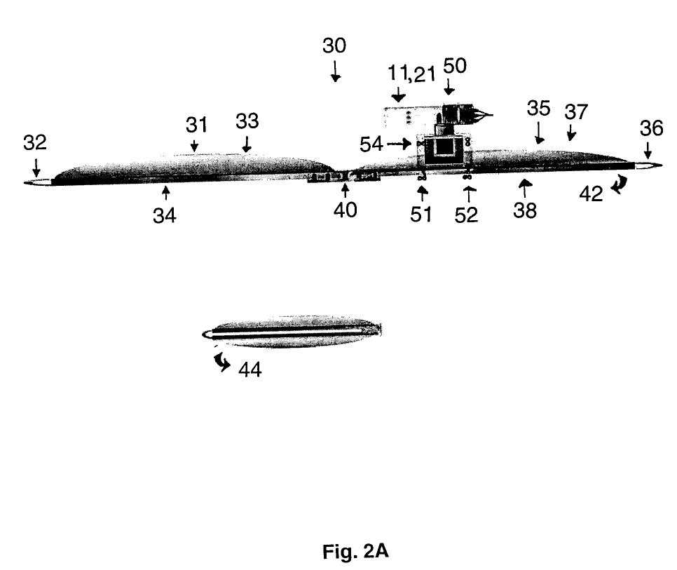

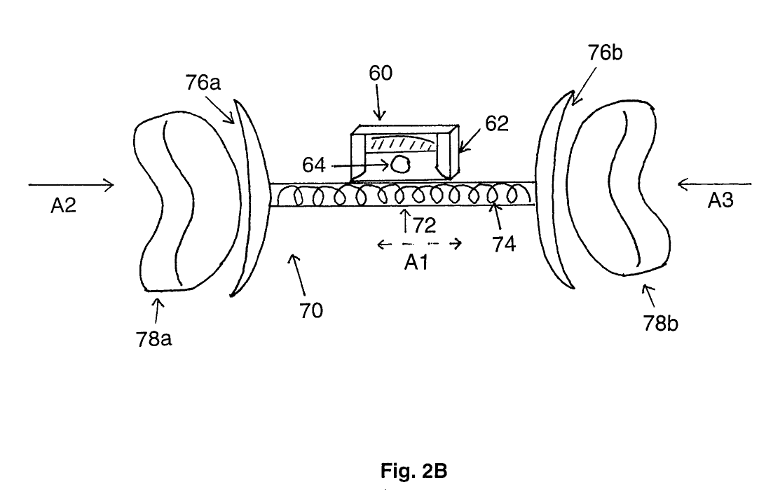

FIG. 2A depicts a hinged folding probe stabilization platform. The platform 30 comprises a first section 31 and a second section 35. Each section 31, 35 has a first end with a handle 32, 36 attached thereto for transport and a second end such that the second ends are attached to each other by a hinge 40. Each section 31, 35 has an upper padded surface 33, 37 and a lower plexiglass platform base 34, 38 to which the padded surface 33, 37 is attached.

The second section 35 further comprises a probe holder 50. The holder 50 is disposed on the upper padded surface 37 and fastened at a lower surface to the plexiglass base 38 via recessed wing nuts 51, 52. The upper surface of the probe holder 50 has a restraining or locking means 54 with which to detachably and rigidly mount the probe 11, 21 to the platform 50. During use, the probe, either 11 or 21, is locked into a holder 50 mounted on an examination platform 30. The holder prevents the probe from moving during exercise or electrical stimulation.

The hinged folding platform 30 provides portability. The probe holder 50 attaches to the platform 30 and locks the probe 11, 12 in place once it has been properly placed. The holder, therefore, allows for adjustment of tilt and penetration prior to locking in place such that the probe is stabilized during contractions.

The second section 35 moves in direction 42 to close the platform such that the surface of the lower platform base 38 is disposed adjacent and in parallel relation to the surface of the lower platform base 34 of the first section 31. In such arrangement the handles 32, 36 are also adjacent each other for carrying purposes. To open the platform 30 prior to attachment of the holder 50 thereto the second section 35 is moved in a direction 44. To maintain a flat surface, the hinge 12 mechanism is restricted to 180 degrees of angular travel. This feature will permit use of the platform on a conventional padded examination table while insuring that the probe holder will not move as the subject shifts position. Additionally, the platform may be mounted to a wall when the patient is standing.

FIG. 2B is a diagram of a multiposition probe stabilizer 60. The probe base connection housing 62 comprises a connection 64 to the probe base (not shown) and contains two axis position sensors (not shown) to provide for detection of angular deflection of the probe during exercise and provides for electrical connection to the pressure transducers and stimulation electrodes.

The probe stabilizer 60 further comprises a calibrated spring connected to the probe (not shown) in the probe base connection housing 62. The probe stabilizer 60 may be attached to a holder 70 as a means of restraint. The holder 70 comprises a cross-member 72 to which the connection housing may be attached and has a spring 74 contained therein to provide resistance to compression for increased stability and support of the base probe connection housing 62. Two support structures 76a,b are attached at either end of the cross-member 72 against which the inner thighs of a patient are positioned. Removable pads 78a,b, e.g., butterfly stabilizer wing pads, may be disposed between a patient's inner thighs and the support structures 76a,b for patient comfort and infection control.

Spring-loaded resistance or release of resistance directionally along an axis defined by A1 stabilizes the connection housing 62 and the probe against probe displacement from a reference axis along the long axis of the probe (not shown). The patient stabilizes the probe in the probe base connection housing 62 by pressing the inner thighs directionally along A2 and A3 against the support structures 76a,b or using body weight to stabilize the probe when the probe stabilizer is positioned as such. A probe (not shown) so stabilized may be used on a patient in a supine position, lying on her side or in a standing position.

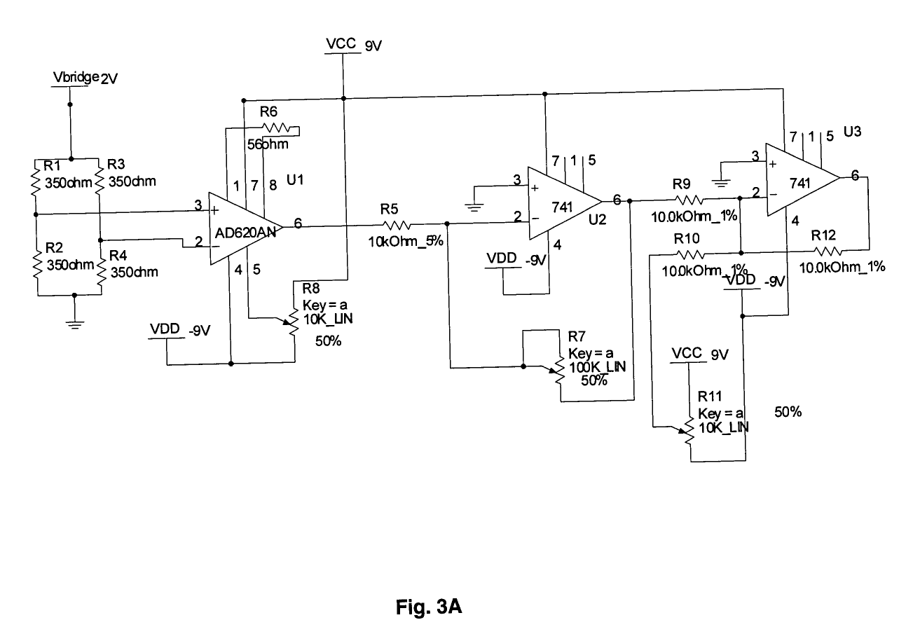

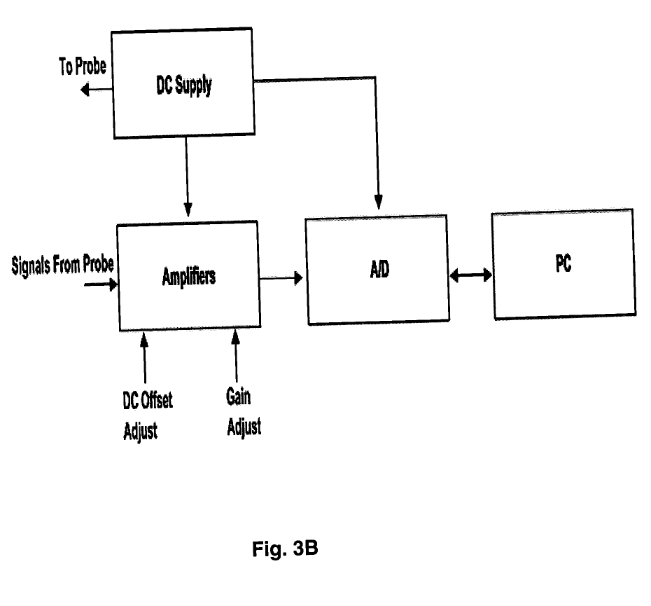

FIG. 3A is a schematic of a differential instrumentation amplifier used to amplify the signal received from the force transducers (not shown). The non-disposable section of the probe contains these differential instrumentation amplifiers. An example of such an amplifier is the Analog Devices AD620. Other suitable differential instrumentation amplifiers with adequate gain, common-mode rejection ratio, and input leakage current may be used in this invention. Mounting of these amplifiers near the transducers improves signal to noise and common-mode rejection performance. Thus, the force signals sent along the cable to the interface are on the order of 100-200 mV rather than 0.1-0.5 mV.

The force proportional output signal is taken from pin 6 of the 741 (or similar) operational amplifier for A/D conversion. One stage is used for each force transducer. The AD620 stage is located on the main body of the probe while the 741 (or similar) stage is located in the interface unit. R7 and R8 are offset adjustments for each amplifier state; for example, R7 would be used primarily when the system is used on subjects. R10 is the DC offset adjustment.

With continued reference to FIG. 3A, FIG. 3B depicts an interface block diagram. A second stage of amplification is provided in the interface in order to fully utilize the dynamic range of the A/D system and to provide for variable gain (for calibration) and DC offset adjustment. This interface unit contains the second stage of amplification, controls for gain and DC offset, the A/D conversion system, and connectors for the computer and probe.

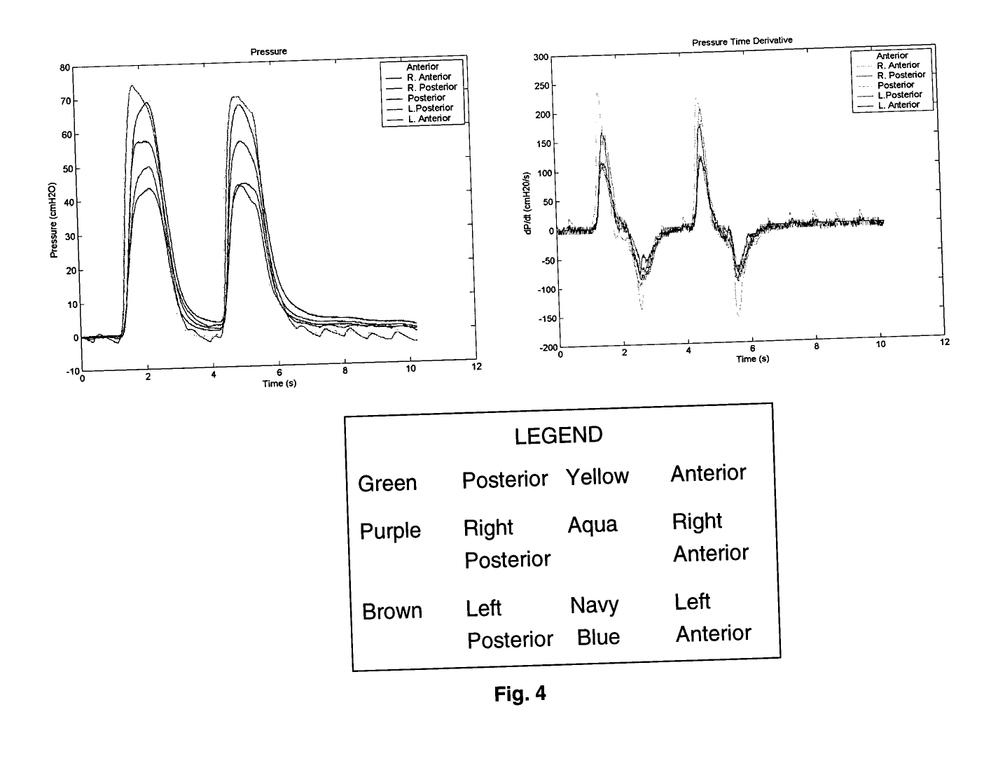

FIG. 4 depicts time-pressure curves and time derivatives that are produced for each transducer thereby providing indication of regional force development. Pressure is shown in units of cm H₂O which is a customary unit of pressure in physiologic measurements. Pressure can be shown in any desired unit by application of the appropriate conversion factor. During exercise, each force signal is digitized at an adequate rate, e.g., 100 Hz. Signal acquisition begins prior to contraction and continues until after the subject is instructed to relax. For force measurement during electrical stimulation, signal acquisition begins prior to delivery of stimulation and continues until after the stimulation is turned off. These time-pressure curves correlate voltage, which is linearly proportional to pressure with known proportionality, to time in seconds.

Also depicted in FIG. 4 are the time derivatives of the time-pressure curves. In this example cm H₂O per second are measured as a function of time. This corresponds linearly to the rate of contraction or relaxation. Contraction effort in progress is indicated by positive values and relaxation in progress is indicated by negative values. A value of zero corresponds to constant pressure.

Analyses for each channel include, but are not limited to, peak force, time to peak force, rate of force development, stability of force maintenance, rate of relaxation, and total effort. These analyses are available for voluntary contraction effort, and for electrical stimulation induced muscle response.

The following references are cited herein.

1. Kegel A H. (1948). Progressive resistance exercise in the functional restoration of the perineal muscles. American Journal of Obstetrics and Gynecology, 56: 238-248.

2. Jones E G & Kegel A H. (1952). Treatment of urinary stress incontinence with results in 117 patients treated by active exercise of pubococcygei. Surgery, Gynecology, and Obstetrics, 94: 179-188.

3. Kegel A H. (1951). Physiologic therapy for urinary stress incontinence. Journal of the American Medical Association, 146:915.

4. Kegel A H. (1956). Stress incontinence of urine in women: Physiologic treatment. Journal, International College of Surgeons, 25: 487.

5. Kegel A H. & Powell T O. (1950). The physiologic treatment of urinary stress incontinence. Journal of Urology, 63: 808-814.

6. Gilpin S A, Gosling J A, Smith A R B, & Warrell D W. (1989). The pathogenesis of genitourinary prolapse and stress incontinence of urine: A histological and histochemical study. British Journal of Obstetrics and Gynecology, 96: 15-23.

7. Wells T J. (1990). Pelvic (floor) muscle exercise. Journal of the American Geriatric Society, 38(3): 333-337.

8. Dougherty M, Bishop K, Mooney, R, Gimotty P, & Williams B. (1993). Graded pelvic muscle exercise: Effect on stress urinary incontinence. Journal of Reproductive Medicine, 38(9): 684-691.

Any patents or publications mentioned in this specification are indicative of the levels of those skilled in the art to which the invention pertains. These patents and publications are herein incorporated by reference to the same extent as if it was indicated that each publication was incorporated specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A luminal force measurement device to assess pelvic floor muscle function for diagnosis or for treatment of an individual comprising:
a cylindrical probe having a section insertable into a body lumen, wherein said insertable section is disposable or is non-disposable, and a non-disposable section connected thereto;
a plurality of force transducers;
a plurality of stimulation electrode pairs;
a plurality of differential instrumentation amplifiers; and
a means of holding said probe in position within the body lumen.

2. The luminal force measurement device of claim 1, further comprising depth index markings on said disposable insertable section or on a sheath covering said non-disposable insertable section indicating depth of insertion into said body lumen.

3. The luminal force measurement device of claim 1, wherein said insertable section of said probe comprises a rigid or a compliant material.

4. The luminal force measurement device of claim 1, further comprising:
an interface unit, said unit comprising
a second stage of force transducer signal amplification;
gain and DC offset adjustment;
electrical stimulation signal generation;
analog-to-digital conversion; and
means for connecting said interface to a control computer.

5. The luminal force measurement device of claim 4, wherein said interface unit is battery powered and the control computer is a battery powered portable computer.

6. The luminal force measurement device of claim 4, wherein said means for connecting said probe to the control computer is via a standardized serial port, a standardized parallel port, a standardized wired communication path, or a standardized wireless communication path.

7. The luminal force measurement device of claim 1, wherein said means to hold the probe in position comprises:
an examination platform; and
a probe holder, wherein said probe holder is detachable from said examination platform.

8. The luminal force measurement device of claim 7, wherein the non-disposable section of said probe connects to the probe holder.

9. The luminal force measurement device of claim 7, wherein said examination platform comprises:
a first section;
a second section, comprising said detachable probe holder;
a handle at a first end of each of said first and second sections; and
a hinge connecting a second end of each of said first and second sections.

10. The luminal force measurement device of claim 9, wherein said hinged sections fold into an adjacent and a parallel relation such that said examination platform may be carried by said handles.

11. The luminal force measurement device of claim 9, wherein an upper surface of said first and second sections is padded.

12. The luminal force measurement device of claim 7, wherein said probe holder is detached from said examination platform prior to folding said platform.

13. The luminal force measurement device of claim 7, wherein said examination platform is placed on a bed, on an examining table or mounted to a wall for use.

14. The luminal force measurement device of claim 7, wherein prior to securing said probe within said probe holder, said probe is movable within said probe holder to adjust tilt and penetration of said probe within the individual.

15. The luminal force measurement device of claim 1, wherein said means to hold the probe in position is a multiposition probe stabilizer, comprising:
a probe base connection housing having a connector for said probe; and
axial displacement sensors connected to said probe to detect displacement of said probe from an axis along the length of said probe, said probe inserted within a body lumen.

16. The luminal force measurement device of claim 15, wherein the non-disposable section of said probe connects to the connector in said probe base connection housing.

17. The luminal force measurement device of claim 15, said probe base connection housing further comprising:
a calibrated spring system resistant to the axial displacement of said probe within the body lumen.

18. The luminal force measurement device of claim 15, further comprising:
a connection housing holder attachable to said probe base connection housing, comprising:
a cross-member having a spring therein, said connection housing attachable thereto; and
a support structure at each end of said cross-member, said support structure having a surface against which an inner thigh of an individual is positioned.

19. The luminal force measurement device of claim 18, further comprising a removable pad disposed between said supporting structure and the inner thigh of the individual.

20. The luminal force measurement device of claim 1, wherein said force transducers and said stimulation electrode pairs are contained on a disposable sheath, each of said electrode pairs in proximate relationship to one of said force tranducers, said sheath removably covering the insertable section of said cylindrical probe, wherein said insertable section is non-disposable.

21. The luminal force measurement device of claim 1, wherein said force transducers and said stimulation electrode pairs are permanently affixed to the insertable section of said probe, each of said electrode pairs in proximate relationship to one of said force tranducers, wherein said insertable section is disposable.

22. The luminal force measurement device of claim 1, wherein said force transducers and said pairs of stimulation electrodes are disposed annularly in a single row around the insertable section of said probe.

23. The luminal force measurement device of claim 1, wherein said force transducers and said pairs of stimulation electrodes are disposed annularly in multiple rows around the insertable section of said probe.

24. The luminal force measurement device of claim 1, wherein said force transducers are strain gauges, piezoelectric sensors, optical sensors, pneumatic mechanisms, or resistive or pressure sensors.

25. The luminal force measurement device of claim 1, wherein said force transducers have a directional component for measurable radial force.

26. The luminal force measurement device of claim 1, wherein said differential instrumentation amplifiers are located on the non-disposable section of said probe.

27. The luminal force measurement device of claim 1, wherein the tip of said insertable section of said probe is rounded.

28. The luminal force measurement device of claim 1, wherein said probe has a diameter of about 1.5 cm to about 2.5 cm and wherein said force transducers and said stimulation electrodes are positioned about 5 cm from the tip of the insertable section of said probe.

29. The luminal force measurement device of claim 1, wherein said probe has a diameter of about 0.75 cm to about 1.5 cm and wherein said force transducers and said stimulation electrodes are positioned about 2 cm from the tip of the insertable section of said probe.

30. The luminal force measurement device of claim 1, wherein said probe is positioned intravaginally or intrarectally.

31. The luminal force measurement device of claim 1, wherein said probe has a total length of at least 20 cm.

32. The luminal force measurement device of claim 1, wherein said stimulation electrodes are used as electromyography electrodes simultaneously with pressure measurements.

33. A method of assessing pelvic floor muscle function, comprising the steps of:
positioning a probe of the luminal force measurement device of claim 1 within an individual, said probe position intravaginally or intrarectally;
causing pelvic muscles to generate force;
transducing said developed muscular force to an electrical signal;
converting said electrical signal from an analog signal to a digital signal;
transmitting said digital signal to a control computer; and
converting said digital signal to data thereby assessing the pelvic floor muscle function in said individual.

34. The method of claim 33, wherein said force is applied voluntarily by said individual via contraction of the pelvic floor muscles or said force is produced by an electrical stimulus applied to specific regions.

35. The method of claim 34, wherein termination of the contractile force by voluntary relaxation or by cessation of electrical stimulus to the specific regions is monitored to assess quantitatively the rate of relaxation and uniformity in the relaxation rate.
